(19) United States
(12) Patent Application Publication
(10) Pub. No.: US 2012/0029397 A1
(43) Pub. Date: Feb. 2, 2012

(54) TUMOR TREATMENT USING ULTRASOUND CAVITATION

(75) Inventors: Chien Ting Chin, Tarrytown, NY (US); Christopher Stephen Hall, Hopewell Junction, NY (US); Alexander L. Klibanov, Charlottesville, VA (US)

(73) Assignees: THE UNIVERSITY OF VIRGINIA, CHARLOTTESVILLE, VA (US); KONINKLIJKE PHILIPS ELECTRONICS N.V., EINDHOVEN (NL)

(21) Appl. No.: 13/262,924

(22) PCT Filed: Apr. 2, 2010
(86) PCT No.: PCT/IB2010/051456

§ 371 Date: Oct. 5, 2011

Related U.S. Application Data
(60) Provisional application No. 61/169,355, filed on Apr. 15, 2009.

(51) Int. Cl. A61N 7/00

(52) U.S. Cl. 601/2

(57) ABSTRACT

Growth in body tissue is slowed, arrested or reversed. In one aspect, this is accomplished by providing bubbles (S315), and delivering, to cause temporary change in physiology that at least one of retards, arrests and reverses said growth, a series of one or more pulses (206) of energy to respective focal points (213) at the site of the growth. In another aspect, temporary change in physiology, such as transient vasospasm (216) in vasculature of a neoplasm, is induced via the mechanical, non-thermal effects of fluid cavitation caused by the pulses. The bubbles, for facilitating the cavitation, in some embodiments, are afforded by administration of a microbubble agent to the host.

### FIELD OF THE INVENTION

The present invention relates to growth control of body tissue, and more particularly to applying energy to retard, arrest or reverse growth.

### BACKGROUND OF THE INVENTION

Cancers of all forms remain a major killer worldwide. A significant limitation of existing and prospective cancer therapies has been and continues to be toxic side effects.

The heating effect of continuous-wave (CW) focused ultrasound is used to kill cells in the treatment of uterine fibroid. Unfortunately, the extent of the treated area is difficult to control, because blood circulation has a strong local effect on the temperature field that develops in the tissue.

Non-heating approaches involving ultrasonic cavitation are known to cause a range of bioeffects including hemolysis, hemorrhage in the microvasculature, sonoporation, transient opening of the blood brain barrier, apoptosis and cell death. The type and severity of the bioeffects depend on many factors, including ultrasound parameters (frequency, amplitude, duty cycle, etc.), concentration, size and type of microbubble seeds, if any were introduced, and the microenvironment of the cavitation events (tissue type and blood flow).

### SUMMARY OF THE INVENTION

The present invention is directed to overcoming or mitigating the above-described limitations of the prior art.

There have been many proposals to exploit the cavitation effect to deliver drugs or genes to a target zone. Ultrasound mediated drug and gene delivery is a promising approach to treat a number of diseases.

However, these methods are still in the development phase, and one of the unresolved issues is that the drug or genetic molecules released by such methods may be washed away from the treatment site by the blood flow, so that localized release may not result in the ideal distribution of drug molecules in the body. Similarly, the thermal energy deposited by HIFU (high-intensity focused ultrasound) techniques, as mentioned above in connection with the heating effect, is also liable to spread, by direct conduction and blood flow convection.

Recent studies have revealed unique details of tumor neovasculature. Typically, hypoxia of the tumor tissue induces angiogenesis, which allows the tumor to grow rapidly, beyond the oxygen diffusion limit. These new vessels are poorly organized and display numerous deficiencies or abnormalities not found in normal tissues. Since tumor cells generally proliferate until a resource limit is reached, most tumors are perfused at a critical level.

As of now, few approaches exist to exploit the distinct characteristics of tumor vasculature. One such approach, familiar to those skilled in art, is the administration of anti-VEGF (anti-vascular endothelial growth factor) antibody, or other anti-angiogenesis drug candidates, to suppress neovasculature formation. A number of angiogenesis inhibitors are now in clinical development. Another approach is based on the use of the EPR (enhanced permeability and retention) effect, whereby the blood-circulating pharmaceutical agent, usually in the form of a drug carrier nanoparticle, such as liposome, micelle, or a macromolecule/complex, is extravasating in the tumor area, leaving the vasculature and moving into the tissue interstitial space through fenestrations, due to the incomplete junctions between endothelial cells in the tumor vasculature. Several drug delivery products are now available that take advantage of this approach, most widely known being Doxil™ (Caelyx™) long-circulating liposome with doxorubicin anthracycline anticancer antibiotic.

The inventors of the present application have discovered that ultrasound cavitation effects, potentiated by microbubbles, can induce changes in the tumor physiology for extended periods of time and thereby can cause restriction of tumor growth and even regression, with reduction of tumor size.

In accordance with the present invention, acute localized disruption of blood flow is induced to treat neoplasia. This vasospasm effect is sensitized in tumor vasculature and, therefore, normal tissue in the treated zone is spared differentially. In addition, the critical state of tumor perfusion allows a greater probability and severity of ischemia subsequent to flow disruption.

Since cavitation events are short-lived, lasting typically a fraction of a second after the incident ultrasound is turned off, direct bioeffects of cavitation are well localized to the insonation zone.

Test results of the present inventors show the vasospasm effect to be triggered within seconds after the ultrasound treatment, offering capability of direct monitoring in real-time.

As proposed herein, use is made of microbubbles which respond in an ultrasound field by oscillating with stimulation from the sound wave. This oscillation can be controlled to be stable (continuous) and unstable (rapid implosion). These oscillations allow for enhanced interaction of the ultrasound field with tissue through the intermediate effects of the movement of the surface of the microbubble.

Procedurally in accordance with a version of the present invention, retarding, arresting or reversing tissue growth involves providing bubbles for a current site of the growth, and, to cause temporary change in physiology that at least one of retards, arrests and reverses said growth, delivering a pulse of energy to a current focal point in the current site. If, in the current site, a next focal point of a pulse to be delivered exists, the delivering is performed to that next focal point as the current focal point. This is repeated, each time for a next focal point as the current focal point, until there is no next focal point to which a pulse is to be delivered.

As an extension of the above, wherein the providing of bubbles also provides bubbles for a next site of the growth, the delivering of pulses to the focal points at the current site is followed by the above-described point-by-point delivering of pulses to the focal points at the next site.

In one aspect of the invention, body tissue at the current site has vasculature and, in an additional step, a check is made for vasospasm in the vasculature.

In one variation of the above, the vasculature is checked for influx of bubbles to confirm occurrence of vasospasm.

The checking, in an embodiment of the above, is performed where bubbles were destroyed in the immediately preceding pulse. The influx, if it occurs at the time of checking, is therefore an influx of fresh bubbles.

In yet another variation, if, in the checking, vasospasm is not found, and additional focal points in the current site are to be delivered a pulse, the pulsing procedure is repeated for the additional points.

In a yet further variation, the temporary change in physiology includes the vasospasm, and disruption, and any blockage, of blood flow due to the vasospasm is temporary, lasting more than one minute and less than 8 hours.

As a variation on the above, the disruption, and any blockage, results in retention of blood constituents existing in the vasculature upon onset of the vasospasm.

In one version of the invention, the body tissue at the current site having vasculature, the delivering of the first pulse is preceded by detecting bubbles in the vasculature.

In another version of the invention, the procedure may serve as a medical treatment to be repeated over time.

According to an alternative version, the body tissue at the current site having vasculature, the pulse exerts pressure sufficient to cause cavitation in the vasculature, but insufficient to permanently damage blood vessels in the vasculature.

In another version, an agent for facilitating cavitation at the current site as a result of the delivering is administered to the host of the neoplasm.

In a further version, the pulse is an ultrasound pulse focused on the current focal point.

In one aspect, the pulse has a frequency of at least 250 kHz.

In a different aspect, the pulse has a pulse width shorter than 1000 milliseconds.

In a yet different aspect, the tissue growth is neoplasia, the current site being a neoplasm.

Optionally, in accordance with the invention, the neoplasm exceeds 10 millimeters in length.

In a further aspect, the pulse exerts pressure on the current site of amplitude greater than 0.1 megapascals.

In an alternative aspect, exerting control over growth in body tissue entails a cavitation generator for inducing, through mechanical, non-thermal effects of cavitation in fluid, temporary change in physiology that at least one of retards, arrests and reverses the growth. It further entails an agent administrator configured for administering in real time to a host of the growth an agent for facilitating the cavitation.

Further involved with regard to the above alternative, in some embodiments, is real-time monitoring for occurrence of vasospasm in the vasculature of a neoplasm.

In other embodiments, the exertion of control over the neoplasm involves a control system for sequencing the inducing and delivery of bubbles for the inducing, and a bubble monitor for monitoring for presence of bubbles. The sequencing causes performance of the inducing selectively in real time, in conjunction with an outcome of the monitoring for presence of bubbles.

As a further variation, the sequencing selectively causes in real time, in conjunction with an outcome of the monitoring, the administering of the agent for facilitating the cavitation.

In embodiments of the present invention, a device for the exerting control over neoplasm growth is implemented as one or more integrated circuits.

Details of the novel growth control procedure are set forth further below, with the aid of the following drawings.

### BRIEF DESCRIPTION OF THE DRAWINGS

FIG. 1 is a schematic diagram exemplary of a device for exerting growth control over a neoplasm, in accordance with the present invention;

FIG. 2 is a conceptual diagram depicting, as an example, a cross-section of a neoplasm in an organ or blood vessel of a host, and examples of operations and pulse parameter limits utilizable, in accordance with the present invention;

FIG. 3 is a flow chart showing an exemplary procedure for retarding, arresting or reversing neoplasm growth, and an exemplary timeline for repeating the procedure, according to the present invention; and

FIGS. 4A, 4B are graphs of neoplasm size over time in actual studies, in accordance with the present invention.

### DETAILED DESCRIPTION OF EMBODIMENTS

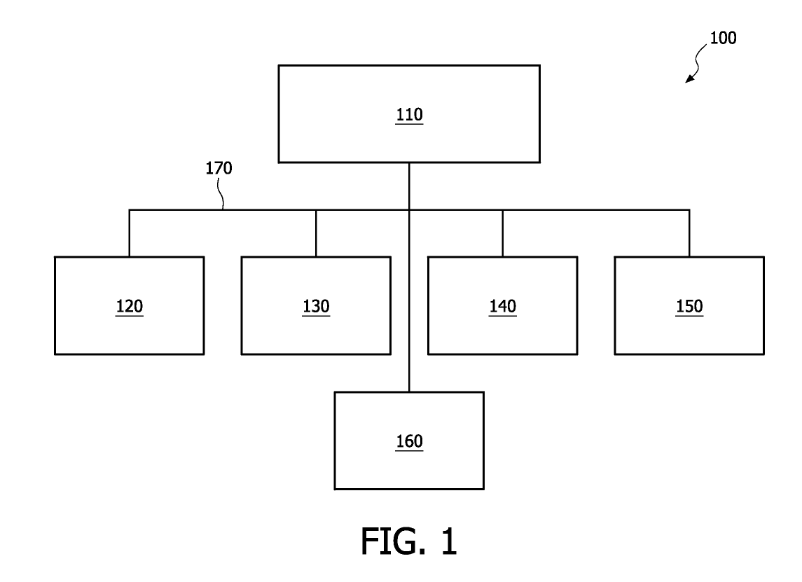

FIG. 1 depicts, by way of illustrative and non-limitative example, a device 100 for exerting growth control over a neoplasm, e.g., tumor. The device 100 includes a control system 110, a cavitation generator 120, a vasospasm monitor 130, a bubble monitor 140, an agent administrator 150, and an imaging system 160, connected on a communication and power bus 170.

The cavitation generator 120 emits pulses of energy to cause cavitation in a fluid, such as in the vasculature of a subject.

The vasospasm monitor 130 monitors for vasospasm in the vasculature being pulsed.

The bubble monitor 140 monitors for the presence of bubbles, e.g., microbubbles, sufficient for effective cavitation.

The agent administrator 150 facilitates cavitation by providing bubbles for the vasculature to be pulsed. Specifically, this may be done by injecting microbubble agent, which is a suspension of microbubbles, into the subject, i.e., the host of the neoplasm. Administration is, for example, via an IV (intravenous) medical device in the arm or forearm.

The imaging system 160 interacts with the cavitation generator 120 to provide guidance based imaging which could be ultrasound, MRI (magnetic resonance imaging) or CT (computed tomography), among other imaging modalities. The imaging system 160 may also be used by the bubble monitor 140 in monitoring for the presence of bubbles. It may also include features for detecting and mapping blood flow, before and after treatment.

It is the function of the control system 110 to coordinate, in real time, the other modules 120-160 so that agent and pulsed energy are delivered at appropriate levels, in synchrony. The control system 110 may comprise a driving system capable of exciting a therapy transducer of the cavitation generator to appropriate pressures. The control system 110 can be configured for determining the optimum rate for injection of microbubbles, timing of ultrasound pulses, and the optimum number or microbubbles in the treatment zone.

In one version, the control system 110 includes a motor system to physically move the cavitation generator 120 to direct the pulsing to different sites.

In another version, the control system 110 includes a driving system which is capable of electronically directing the pulsing to different sites.

Alternatively, the device 100 can be realized, for example, separate from the imaging system, and, as such, may be implemented as one or more integrated circuits for an otherwise pre-existing ultrasound system.

A basic version of the device 100, moreover, is achievable, for example, just with the cavitation generator 120 and the bubble monitor 140, those being inclusive of the appropriate control logic.

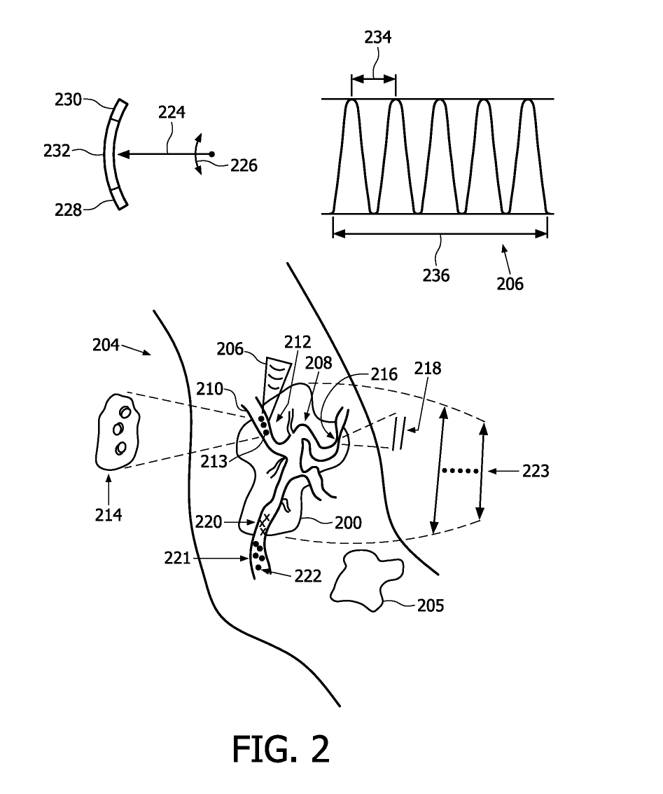

FIG. 2 illustrates, as an example, a cross-section of a neoplasm 200 in an organ, vein or artery 202 of a host 204, and examples of operations and pulse parameter limits utilizable. Another or a next neoplasm 205 is also shown.

The host 204 is a medical subject, such as a human medical patient or an animal, such as a warm-blooded mammal, although the present invention is not limited to any particular living form. The subject could also be a medical sample, in vitro or ex vivo.

An ultrasound pulse 206 is controlled to create bubbles that oscillate with the sound wave, i.e., stable (or non-inertial) cavitation, or to produce unstable (inertial) cavitation in which the oscillation is characterized by rapid implosion or collapse of bubbles.

The neoplasm 200, which is the current site of growth over which control is to be exerted, has vasculature 208 comprised of blood vessels 210 into which microbubbles 212 may be introduced. Injection into the bloodstream of a microbubble agent, along with the flowing motion of the blood, is sufficient to create the microbubbles 212.

Delivery of the pulse 206 to a focal point 213 directly causes the microbubble oscillation 214, shown in expanded view in FIG. 2.

Most of the time, a series of one or more appropriately configured pulses 206 to respective focal points 213 in the neoplasm 200 will trigger vasospasm, i.e., sudden constriction of the blood vessel 210 that reduces or disrupts the blood flow. A number of treatments, which may be conducted sporadically, may be needed to achieve a targeted degree of suppression or reversal of neoplasia.

The vasospasm, which is a change in physiology due to the insonation, and the disruption, and any blockage, of blood flow due to vasospasm, caused by the techniques proposed herein, are temporary, lasting, for instance between 1 minute and 8 hours, and typically lasting for minutes, but long enough to effectively treat neoplasia. A constricted portion 216 of a blood vessel is shown in expanded view as a recovered portion 218, having later recovered from vasospasm. The disruption, and any blockage, results in retention of blood constituents existing in the vasculature upon onset of the vasospasm. If drugs used in medically treating the neoplasm 200 were in the blood at that time, beneficially they are retained locally to provide treatment.

Live-animal experiments, discussed in more detail further below, have shown three related effects: acute reduction of perfusion in the neoplasm, retardation or even reversal of neoplasm growth, and prolonged survival of the test animals. The result is particularly notable on large tumors, i.e., having a length in excess of 10 mm (millimeters), in which a wide variety of known treatments normally fail.

The series of one or more pulses 206 are delivered to the neoplasm 200 typically according to a treatment plan, and then a check is made for vasospasm. Since the pulse 206 destroys the microbubbles 220, shown as x's, any influx of microbubbles 221 is an influx 222 of fresh microbubbles. The microbubbles 221 of the influx 222 can be detected, because they scatter ultrasound to produce a non-specular reflection (or “speckle”), and in Doppler ultrasound, they increase the flow signal from blood vessels.

By checking the site of insonation right after insonation, it can be determined whether vasospasm has occurred. In particular, within seconds after treatment, vasospasm can be confirmed by imaging the blood flow enhanced by microbubble agent.

The microbubbles 220 were destroyed in the immediately preceding pulse 206. So, the influx 222 of fresh bubbles 221 indicates lack of vasospasm 216. This inflow 222 can be easily observed.

If, on the other hand, vasospasm 216 has occurred, its severity and duration can be measured by means of the blood flow imaging. The influx 222, in this case, would not be seen until, typically, several minutes after the last pulse.

Vasospasm that has had the effect of reducing the size, e.g., length, of the neoplasm 200 is illustrated by the series of decreasing, double-headed arrows 223.

Pressure of the pulse 206 is represented conceptually in FIG. 2 by a pointer 224 rotatable as indicated by the curved, double-headed arrow 226. The pointer 224 can conceptually be rotated to indicate a no-cavitation zone 228, a permanent damage zone 230, or, in keeping with aspects of the present invention, an intervening cavitation zone 232. The pressure should not be so low as to fail to cause cavitation. The pressure likewise should be insufficient to permanently damage the vasculature, since such levels could potentially give rise to the incidental tissue damage associated with such a technique. A typical pressure to be exerted incident upon the neoplasm could be 5 megapascals or, for example, at least 0.1 megapascals.

The pulse 206 has a peak-to-peak time 234 of 2 μs (microseconds) or, equivalently, a frequency of 1/(2 μs)=500 kHz (kilohertz). A minimum frequency to support the pulse 206, in accordance with the present invention, may be, for instance, 250 kHz. Although the pulse 206, shown for simplicity of demonstration with only 5 peaks, is shorter than a typical actual pulse, the pulse width 236 may be kept shorter than, for example, 1000 ms (milliseconds).

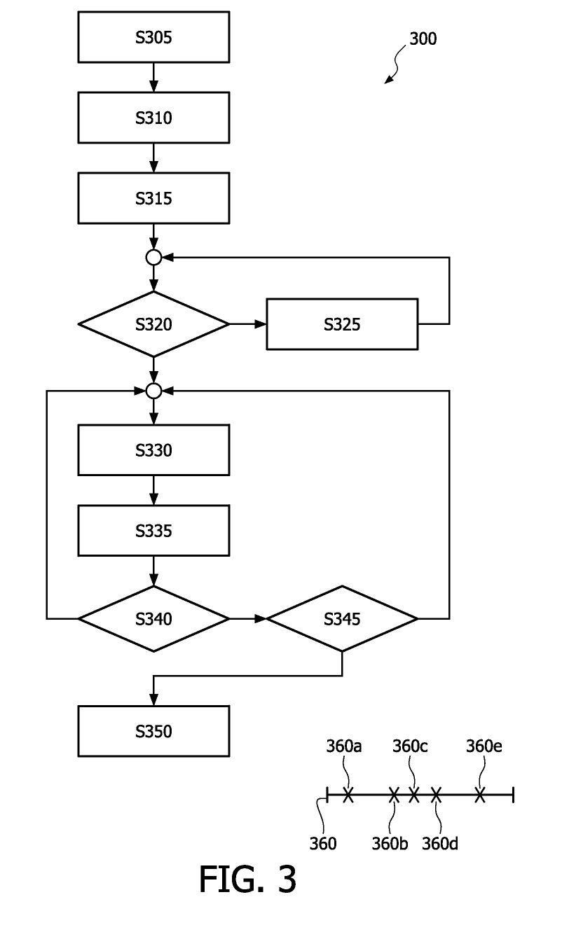

FIG. 3 shows an exemplary procedure 300 for retarding, arresting or reversing neoplasm growth, and an exemplary timeline for repeating the procedure.

A delivery needle or catheter is placed into a vein or artery 202 of the subject 204 (step S305). The therapy transducer is positioned over the treatment zone (step S310). Optionally, guidance by means of the imaging system 160 allows for more accurate placement. Flow of the microbubble agent commences when the user actuates a switch. The agent circulates throughout the host's bloodstream, providing microbubbles 221 for the site 200 of the growth (step S315). When the bubble monitor 140 detects an appropriate amount of microbubbles 212 in the blood vessel 210 of the vasculature 208 (steps S320, S325), the control system 110 is notified. The control system 110 then causes the cavitation generator 120 to emit an ultrasound pulse 206 focused on a current focal point 213 in the neoplasm 200 (step S330). If a next focal point to be pulsed exists (steps S335, S340), as in a treatment plan, processing returns to step S330. Movement, each time, from the current focal point to the next focal point may be mechanical or electronically steered. Otherwise, if a next focal point to be pulsed does not exist (steps S335, S340), the vasospasm monitor 130 commences checking for vasospasm 216 in the vasculature 208. If vasospasm 216 is not found, and additional focal points are to be pulsed in an effort to achieve vasospasm (step S345), processing branches back to step S330. Otherwise, if vasospasm is detected or no additional focal points are to be pulsed (step S345), the procedure is completed (S350).

It should be noted that each of the two branch paths back from step S340 to S330 may be for the processing of a next site, e.g., the next neoplasm 205, for a series of one or more respective focal points. This would apply, in the event microbubbles 212 for the next site, e.g., in an appropriate amount overall or localized, were detected in step S320. Alternatively, the branch back may deliver to the same site 200 pulses 206 of longer or shorter duration, of greater or lesser power, of higher or lower frequency, etc.

A hypothetical scheduling or log timeline 360 shows several repetitions 360a-360e of the procedure 300. The timeline 360 may represent a span of hours, days, months, etc. Temporally, treatments may be scheduled or occur sporadically.

The neoplasm growth control device 100 could be extra-corporeal for non-invasive treatment or mounted on the end of a catheter or needle for minimally invasive treatment. A microbubble delivery system of or operated by the agent administrator 150 could also be needle or catheter based.

An example system that has been used for testing has an ultrasound therapy transducer with 8 annular rings with a diameter of 8 cm (centimeters), focal length of 8 cm (f-number= 8/8=1), center frequency of 1.2 MHz (megahertz). This transducer is excited by a drive system capable of providing pulsed-wave delivery. It was found that, by choosing the excitation appropriately (namely, bursts of high intensity ultrasound), blood flow in the treated region can be temporarily stopped. The system incorporates an ultrasound imaging system which provided real-time monitoring and follow-up assessment capabilities. With the injection of a microbubble agent, the system has very high sensitivity to even very low level of perfusion.

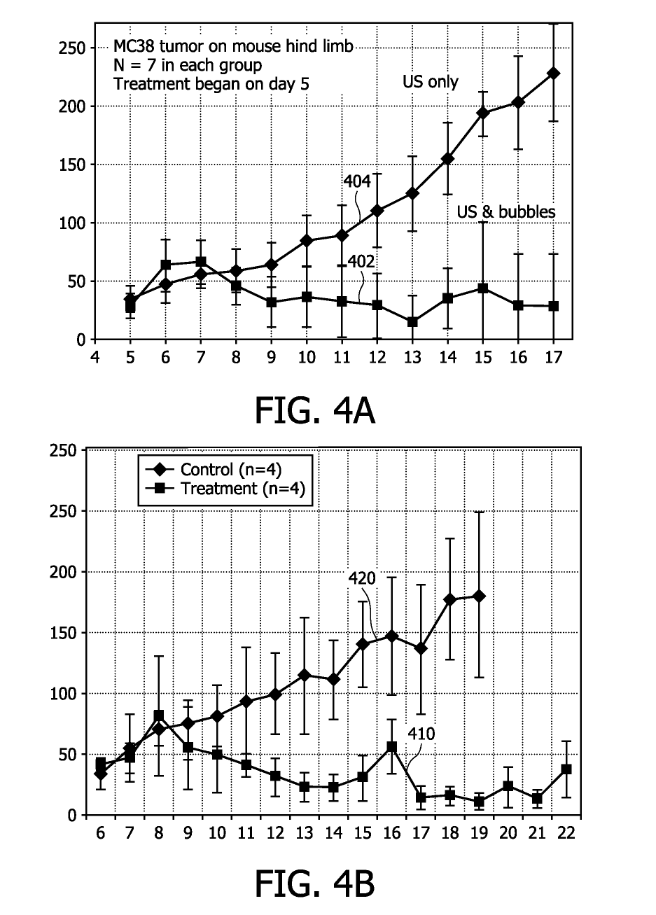

FIG. 4A shows a preliminary result of neoplasm size in an ongoing study, in particular a treated group 402 and a control group 404. The ordinate represents neoplasm size in square millimeters. The abscissa represents neoplasm age in days. The effects of treatment on neoplasm size over time are visible from the graph.

FIG. 4B is second study. As in the first study, the treated group 410 shows retardation of tumor growth in a statistically significant manner. Body weight measurement shows no statistically significant difference between the treated group 410 and control group 420.

In each of the two studies, eventually the control tumors exceeded the size limit established by adopted ethic standards of humane treatment. Accordingly, upon exceeding the prescribed size, members of the control group 420 are euthanized.

### EXAMPLE

Reduction of blood flow in the tumor vasculature after microbubble destruction by ultrasound.

Evaluate tumor blood flow by real-time ultrasound contrast imaging after high-amplitude focused ultrasound treatment in a murine model.

Microbubbles were prepared from perfluorobutane gas and stabilized with a phosphatidylcholine/PEG stearate shell. MC38 mouse colon adenocarcinoma cells (J. Schlom, NIH) were subcutaneously administered in the hind leg of C57BL/6 mice. After the tumor reached >5-6 mm size, anesthetized mice were placed under the focused ultrasound transducer. Intravenous administration of 0.05-0.1 ml microbubbles was performed, immediately followed by 1.2 MHz 5 MPa insonation, delivered to the tumor as ten 1 Hz PRF 100K-cycle pulses (TIPS™ system, Philips). Insonation was repeatedly performed, i.e., on essentially a daily basis, to achieve reduction in tumor size.

Ultrasound contrast imaging during and after insonation was performed with CL15 transducer (HDI5000). An imaging transducer was fixed on the therapeutic transducer to achieve real-time guidance capability. Tissue perfusion was monitored as movement of ultrasound contrast microbubbles through the tumor vasculature.

Ultrasound treatment of tumor under the conditions described resulted in minor tissue temperature increase. Destruction of microbubbles during TIPS™ insonation was observed in the tumor vasculature by ultrasound contrast imaging. TIPS™ insonation of microbubbles in the tumor vasculature resulted in an immediate reduction of blood flow in the tumor, as observed by ultrasound contrast imaging. This “vascular stunning” effect was transient; blood flow within the tumor would resume within minutes. In the absence of microbubbles, tumor blood flow was not altered by insonation. Microbubble destruction by diagnostic ultrasound imaging did not cause stunning.

Destruction of microbubbles by high-amplitude therapeutic ultrasound within the tumor vasculature results in the transient reduction of blood flow. This effect may be critical for the success of the ultrasound-assisted tumor drug delivery: in case the blood flow is reduced after the onset of insonation, the drug carrier systems will be unable to reach the tumor. On the other hand, this effect might be exploited to enhance retention of released drugs in the tumor.

Physiological effects of the microbubble destruction in the tumor vasculature by therapeutic ultrasound may have significant influence on the blood flow and tumor perfusion.

Growth in body tissue is slowed, arrested or reversed. In one aspect, this is accomplished by providing bubbles, and delivering, to cause temporary change in physiology that at least one of retards, arrests and reverses said growth, a series of one or more pulses of energy to respective focal points at the site of the growth. In another aspect, temporary change in physiology, such as transient vasospasm in vasculature of a neoplasm, is induced via the mechanical, non-thermal effects of fluid cavitation caused by the pulses. The bubbles, for facilitating the cavitation, in some embodiments, are afforded by administration of a microbubble agent to the host.

Embodiments of the current invention are especially useful for treatment of localized and vascularized tumors such as the liver, breast, prostate, brain, pancreas or other organs addressable by ultrasound. It is within the intended scope of the present invention that it be applied in the treatment of a wide range of cancers.

In accordance with the present invention, the delivered medical treatment is reliable, spatially accurate and of short duration. Adverse bioeffects are avoided or confined locally to the neoplasm. Success of the treatment is immediately verifiable, and even large neoplasms are treatable. As an ultrasound solution, it is low-cost.

It should be noted that the above-mentioned embodiments illustrate rather than limit the invention, and that those skilled in the art will be able to design many alternative embodiments without departing from the scope of the appended claims. For example, the steps S320 through S350 in FIG. 3 can all be performed manually, or automatically without the need for user intervention. Or, some of the steps may be manual and the rest automatic. In the claims, any reference signs placed between parentheses shall not be construed as limiting the claim. Use of the verb “to comprise” and its conjugations does not exclude the presence of elements or steps other than those stated in a claim. The article “a” or “an” preceding an element does not exclude the presence of a plurality of such elements. The invention may be implemented by means of hardware comprising several distinct elements, and by means of a suitably programmed computer having a computer readable medium. The mere fact that certain measures are recited in mutually different dependent claims does not indicate that a combination of these measures cannot be used to advantage.

What is claimed is:

1. A method for retarding, arresting or reversing tissue growth, comprising the acts of:
a) providing bubbles (222) for a current site of said growth;
b) to cause temporary change in physiology that at least one of retards, arrests and reverses said growth, delivering a pulse (206) of energy to a current focal point in said current site; and
c) if, in said current site, a next focal point of a pulse to be delivered exists (S340), repeating the acts b) and c) for said next focal point as said current focal point.

2. The method of claim 1, wherein the act a) comprises providing bubbles for a next site of said growth, said method further comprising, after the act c), repeating the acts b) and c) for said next site as said current site (S345).

3. The method of claim 1, body tissue at said current site having vasculature (208), said method further comprising the act of:
d) checking for vasospasm (216) in said vasculature.

4. The method of claim 3, said checking of the act d) comprising checking said vasculature for influx (222) of bubbles to confirm occurrence of vasospasm.

5. The method of claim 4, said checking being performed where bubbles were destroyed in an immediately preceding pulse of the act b), said influx, if it occurs at a time of said checking, being an influx of fresh bubbles (221).

6. The method of claim 3, further comprising the act of:
e) if vasospasm is not found in the act d), and additional focal points in said current site are to be delivered a pulse in the act b) (S345), repeating the acts b) through d) for said additional focal points.

7. The method of claim 3, said temporary change in physiology comprising said vasospasm, disruption, and any blockage, of blood flow due to said vasospasm being temporary, lasting more than one minute and less than 8 hours.

8. The method of claim 7, said disruption (216), and any blockage, resulting in retention of blood constituents existing in said vasculature upon onset of said vasospasm.

9. The method of claim 1, body tissue at said current site having vasculature, said method further comprising, before the act b), detecting (S320) bubbles in said vasculature.

10. The method of claim 1, as a medical treatment to be repeated over time (360).

11. The method of claim 1, body tissue at said current site having vasculature, said vasculature comprising a blood vessel (210) to which said pulse is delivered in the act b), said pulse in the act b) exerting pressure (232) sufficient to cause cavitation in said vasculature, but insufficient to permanently damage said blood vessel.

12. The method of claim 1, further comprising administering (S315), to a host of said growth, agent for facilitating cavitation at said current site as a result of said delivering.

13. The method of claim 1, said pulse in the act b) being an ultrasound pulse focused on said current focal point (213).

14. The method of claim 1, said pulse in the act b) having a frequency (234) of at least 250 kHz.

15. The method of claim, said pulse in the act b) having a pulse width (236) shorter than 1000 milliseconds.

16. The method of claim 1, said tissue growth being neoplasia, said current site being a neoplasm (200).

17. The method of claim 16, said neoplasm exceeding 10 millimeters in length (223).

18. The method of claim 1, said pulse in the act b) exerting pressure incident on said current site of amplitude greater than 0.1 megapascals.

19. A device for exerting control over growth in body tissue, comprising:
a cavitation generator (120) configured for inducing, through mechanical, non-thermal effects of cavitation (214) in fluid, temporary change in physiology that at least one of retards, arrests and reverses said growth; and
an agent administrator (150) configured for administering in real time to a host of said growth an agent for facilitating said cavitation.

20. The device of claim 19, further comprising a vasospasm monitor (130) configured for real-time monitoring for occurrence of vasospasm in vasculature of a neoplasm.

21. The device of claim 20, further comprising a control system (110) for sequencing said inducing and delivery of bubbles for said inducing, and a bubble monitor (140) for monitoring for presence of bubbles, said sequencing causing performance of said inducing selectively in real time, in conjunction with an outcome of said monitoring for presence of bubbles (S320).

22. The device of claim 21, said sequencing selectively causing in real time, in conjunction with an outcome of said monitoring, said administering.

23. The device of claim 19, implemented as one or more integrated circuits (100).

24. A computer software product for retarding, arresting or reversing tissue growth, comprising a computer readable medium embodying a computer program that includes instructions executable by a processor for:
a) providing bubbles (222) for a current site of said growth;
b) delivering a pulse (206) to a current focal point in said current site; and
c) if, in said current site, a next focal point of a pulse to be delivered exists (S340), repeating the acts b) and c) for said next focal point as said current focal point.
